(19) United States
(12) Patent Application Publication
(10) Pub. No.: US 2010/0247456 A1
(43) Pub. Date: Sep. 30, 2010

(54) METHODS AND COMPOSITIONS FOR PREVENTING CARIES

(76) Inventors: Richard Niederman, Boston, MA (US); Aronita Rosenblatt, Pernambuco (BR)

Correspondence Address: ANTOINETTE G. GIUGLIANO, P.C.;DBA AGG Intellectual Property Law, 100 Cummings Center, Suite 213C, Beverly, MA, 01915, US

(21) Appl. No.: 12/726,059

(22) Filed: Mar. 17, 2010

Related U.S. Application Data
(60) Provisional application No. 61/161,389, filed on Mar. 18, 2009.

(51) Int. Cl. A61K 8/21; A61Q 11/00

(52) U.S. Cl. 424/52

(57) ABSTRACT

The present invention relates to an anti-caries composition that includes Silver Diamine Fluoride (SDF) and a carrier, and methods for using the composition. In particular, the composition includes SDF in an amount between about 1% and about 40% by weight; and a carrier (e.g., natural adhesive, a synthetic adhesive, a drying adhesive, an emulsion adhesive, silicone, copal, glass ionomer, cyanoacrylate, composite resin, latex, epoxy, silicone, polyurethane, denture adhesives, or variants thereof or any combination thereof.

The methods of the present invention relate to arresting caries or reducing the number of active caries lesions. The steps include applying the anti-caries SDF composition to one or more tooth surfaces for about 1-5 minutes, and rinsing the composition from the tooth surface. Another method of the present invention relates to applying a SDF composition to the tooth surface, curing the composition, to thereby treat caries. Caries is arrested or the number of active caries lesions is reduced, as compared to an individual whose tooth surface is not subjected to the SDF composition.

### RELATED APPLICATION

This application claims the benefit of U.S. Provisional Application No. 61/161,389, entitled “Methods and Compositions for Preventing Caries,” by Richard Niederman and Aronita Rosenblatt, filed Mar. 18, 2009.

The entire teachings of the above application are incorporated herein by reference.

### BACKGROUND OF THE INVENTION

Dental caries is a bacterial infection whose metabolic acid byproducts etch, decalcify, and ultimately cause tooth cavitation, or dental decay.

Dentists and patients use fluoride to prevent caries in children and adults. However, despite application of a systemic fluoride (e.g., water fluoridation) or topical fluoride (e.g., gel, varnish, toothpaste) many children and adults continue to get carious lesions (e.g., cavities).

A need exists for a composition that better prevents formation of carious lesions, and treats existing carious lesions. A further need exists for a composition that is easy to apply, and that adheres to the tooth surface without affecting the oral mucosa and other oral structures.

### SUMMARY OF THE INVENTION

The present invention relates to methods for arresting caries or reducing the number of new or active caries lesions in an individual by administering silver diamine fluoride (SDF) in a sustained release composition. The steps of the method include applying a SDF gel composition to the tooth surface of the individual for a period of between about 1 and 5 minutes, optionally rinsing said composition from the tooth surface.

In another embodiment, the methods include applying a SDF varnish composition to the tooth surface of an individual. The varnish is allowed to dry or is cured to adhere to the tooth surface. The varnish, once dried or cured, adheres to the tooth surface for a longer period of time (e.g., 5 minutes to 5 years) and provides sustained release of SDF.

The composition used in the method comprises SDF in an amount of between about 1% and 40% by weight (e.g., about 5% and about 15% by weight), and a varnish, adhesive or gel formulation that provides a sustained release of SDF over time and adheres to the tooth surface. Examples of carriers that can be used include copal, denture adhesive, epoxy, silicone, latex, glass ionomer, composite resin, natural adhesives (e.g., in organic mineral sources, biological sources such as starch, national resins such as casein, bioadhesive mixtures such as gelatin and starch), synthetic adhesives (e.g., elastomers, thermoplastics, emulsions based on polyvinyl acetate, epoly, water based polyurethane, cyanoacrylate), drying adhesives, emulsion adhesives (e.g., polyvinyl acetate) or any combination thereof, or variants thereof.

In an embodiment, the carrier that allows the composition to adhere to the tooth enamel for a period of between about 1 to about 5 minutes can consist of a gel or varnish that can be made from a natural adhesive, a synthetic adhesive, a drying adhesive, an emulsion adhesive or any combination thereof. In the case of a gel, the composition can be used for a short term periods in which the gel is applied to one or more teeth for a period of about 1 to about 5 minutes, and then wiped off or removed. In this embodiment, the composition is applied to teeth for a short period of time and SDF is absorbed by the tooth enamel or dentin for the prevention or treatment of caries. In the case of a varnish, the composition of the present invention can be applied, and the composition can dry or be cured to remain on one or more teeth for a longer period of time (e.g., between about 5 minutes to 5 years). In this case, the composition of the present invention provides a sustained release over a longer period of time to prevent the development of caries, or to treat carious lesions.

The composition of the present invention can include carriers that are natural adhesives. Natural adhesives include adhesives made from inorganic mineral sources or biological sources such as vegetable matter, starch (e.g., dextrin), natural resins or from animals e.g. casein or animal glue, or bioadhesive mixtures such as gelatin and starch. Synthetic adhesives can include adhesives such as elastomers, thermoplastics, emulsion or thermosetting adhesives based on polyvinyl acetate, epoxy, polyurethane, cyanoacrylate polymers or any combination thereof. Drying adhesives can include adhesives made from a mixture of ingredients, such as polymers dissolved in a solvent. Emulsion adhesives can consist of dispersions based on polyvinyl acetate.

As a result of carrying out the method, caries is arrested or the number of active or new caries lesions is reduced, as compared to an individual whose tooth surface is not subjected to the SDF composition, or are subjected to other caries inhibiting agents (e.g. fluoride varnish or rinse, or fluoride toothpaste or gel, or providone iodide, or Ag, all with or without a carrier). In an aspect of the invention, caries is arrested by at least a range of between about 5% and about 30% (e.g., about 10% and about 25%). In an embodiment, the number of active caries lesions is reduced in a range between about 5% and about 30% (e.g., about 10% and about 25%). In certain instances, both caries is arrested and active carious lesions are reduced. In such cases, both are reduced at least by a range between about 30% and about 90% (e.g., about 50% and about 70%).

In yet another embodiment of the present invention, methods for preventing or reducing caries in one or more teeth of an individual are provided. The steps include applying a composition to the tooth surface of the individual for a period of between about 1 and 5 minutes, and rinsing said composition from the tooth surface. Alternatively, the steps include applying the composition to one or more tooth surfaces, and allowing the composition to dry or to be cured. The composition comprises SDF in an amount of between 1% and 40% by weight, and a carrier that provides a sustained release of SDF and adheres to the tooth surface. Caries in one or more teeth of the individual is reduced, as compared to an individual whose tooth surface is not subjected to the SDF composition or other caries inhibiting agent, as described herein. The SDF composition, in an aspect, adheres to the tooth surface and not to the oral mucosa. In certain instances, the methods are provided to individuals that have indications for developing dental caries (e.g., the individual has braces, has x-ray results showing a decrease in enamel density).

The present invention further relates to compositions that include SDF in an amount between about between about 1% and about 40% by weight (e.g., about 5% and about 20% by weight); and a carrier. The composition embodies a sustained release of SDF and adheres to the tooth surface.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A is a clinical photograph prior to the application of silver diamine fluoride. FIG. 1A shows interproximal caries lesions in maxillary incisors of a 5-year-old girl.

FIG. 1B is a clinical photograph following a 60-second application of a 12% silver diamine fluoride. Note that only the caries lesion, not the tooth, is stained.

### DETAILED DESCRIPTION OF THE INVENTION

A description of preferred embodiments of the invention follows.

The present invention relates to methods and compositions for treating or preventing caries in an individual. In particular, the present invention includes applying a composition of Silver Diamine Fluoride (SDF) to enamel and/or dentin to prevent caries, or reduce the incidence or extent of caries.

Dental caries, commonly referred to as tooth decay, is a condition in which damage to the tooth structure occurs. Dental caries is a cavity that is caused by acid release from a bacterial infection, adherent to the teeth. The tooth includes, in part, the enamel, the dentin and the pulp. The enamel comprises the outer surface of the tooth, and the dentin is the layer just below the enamel. The pulp is the central part of the tooth, which includes soft connective tissue, blood vessels and nerves. Caries, as used herein, refers to destruction or decay of the enamel, dentin and/or pulp or any combination thereof. Carious lesions refer to injury to the tooth structure that is caused by caries infection.

The presence, absence or state of caries can be determined by a health professional or lay person using methods that are known in the art. For example, early caries is determined by a visual identification of “white spot” lesions. Caries is also determined by visual and tactile exam identifying discolored or decalcified pits and fissures. Frank cavitation is identified as a clear break in the enamel. The presence of white spots, discolored or decalcified pits and fissures, or frank cavitation indicates the presence of dental caries. Inspection of visible tooth areas can be performed with a dental mirror and explorer. Caries can be identified by its texture and architecture. Healthy enamel and dentin are more dense to probing with a dental instrument, as compared to enamel and dentin that are infected with caries. Additionally, caries can be diagnosed with use of X-rays, especially in areas that are not easily visible. Other technologies such as fiberoptic illumination, lasers and dyes can also be used to identify the presence or absence of caries.

The present invention relates to the “prevention” and/or “reduction” of caries in an individual. Preventing caries refers to stopping active caries, delaying the onset of caries, reducing the occurrence of new caries in an individual, or reducing the number of specific oral bacteria, or changing the types of oral bacteria, or changing the relative number of bacteria. Caries can be prevented or reduced in several ways. For example, the number of teeth having caries in an individual can be reduced (e.g., the reduction in new occurrences of caries). Additionally, a reduction in the volume of caries progression can also occur. The volume of caries infection encompasses the depth of the carious lesion, the number of tooth surfaces and layers infected, and the size of the carious lesion. As such, “reducing” caries infection or the incidence of caries refers to a decrease in one or more of the following: the number of carious lesions of the individual, the depth of the carious lesion, the tooth layers infected, and the size of the carious lesion. A patient that is subjected to the SDF composition of the present invention can be compared with a patient that is not subjected to the composition to determine if a reduction in caries has occurred, for use as a control. Additionally, a reduction in caries can be determined by comparing the state of caries before and after treatment. Improvement in one or more of the characteristics of caries or carious lesions indicates a reduction or treatment of caries. Additionally, the improvement of caries can be determined by a reduction in oral bacterial number, type, or relative number.

The present invention further includes treating caries. “Treating” caries refers to the cessation or reduction of progression of caries. Treating caries includes preventing the carious lesion from getting worse. For example, the carious lesion is treated when the lesion does not get larger in size and/or does not further affect additional tooth structure (e.g., penetrate from the enamel to the dentin).

Active caries refers to caries that is actively creating a lesion. Arrested caries refers to caries that has ceased its progression and is no longer creating a lesion. In an embodiment, the present invention relates to reducing the number of active caries instances in an individual treated with the SDF composition of the present invention, as compared to an individual that has not been treated with SDF. In another embodiment, the present invention includes inducing or increasing arrested caries in an individual treated with SDF composition of the present invention, as compared to an individual without such SDF application, or with a caries inhibiting agent. In a study described in the Exemplification, 16% of children treated with SDF had active caries, as compared with 97% of children treated with traditional fluoride varnish. Similarly, arrested caries was found in 23% of children treated with SDF, as compared with 0% of fluoride varnish treated children. Treatment and prevention of caries is clearly demonstrated by the results described in the Exemplification.

The present invention includes methods for treating an “individual” or a “patient” and the terms are used interchangeably herein. The methods and compositions of the present invention can be used on any individual in whom caries is to be prevented or reduced. Children, between the ages of 4 years and 18 years are generally more susceptible to carious lesions, as compared to adults. As such, an embodiment of the present invention includes applying the composition of the present invention to individuals having an age between about 4 and 18 years.

The composition of teeth (enamel, dentin, and pulp) is similar for children and adults, as the adult teeth begin emerging at 6 years old, and if kept intact remain with the individual for life. Therefore SDF is equally applicable to adults.

Similarly, individuals with braces are more likely to get caries, or those with X-rays results showing a decrease in enamel density. Accordingly, the present invention encompasses identifying a patient that is susceptible to caries and applying the SDF composition of the present invention, as described herein.

Similarly, individuals that are susceptible to caries include those with salivary disorders or reduction in salivary flow or alteration in salivary contents resulting from: heritage, or Sjogrens Syndrome, or chemotherapy, or radiation therapy, or medication (e.g., for heart disease, or psychiatric disorders).

Similarly, individuals with enamel hypoplasia or dentinogenesis imperfecta, or other enamel of dentinal disorders are more likely to get caries.

A composition of SDF is applied to the surface of one or more teeth in an individual. The composition includes between 1% and 40% by weight of SDF, and preferably between 5% and 20% by weight. In an embodiment, a composition of 12% by weight of SDF was effectively used to reduce the occurrence of caries. The SDF gel composition of the present invention includes a, adhesive, or gel that allows the composition to adhere to the tooth enamel for a period of between about 1 to about 10 minutes (e.g., preferably between about 2 and about 5 minutes). In an embodiment, the SDF composition is applied to the tooth surface for about 3 minutes. Additionally, the varnish composition also includes between 1% and 40% by weight of SDF (e.g., between 5% and 20% by weight) and a varnish carrier. Varnishes are compositions that allow one to apply the composition to one or more teeth, and the composition dries or can be cured so that the composition adheres to the teeth for a longer period of time (e.g., 5 minutes to a week to 5 years, and preferably 1 week to 1 year, 2 years, 3 years, etc.) Drying agents such as alcohol can be used for a various composition. The composition can be a viscous compound that allows the composition to adhere to the tooth enamel or dentin, in another embodiment, without spreading to other oral surfaces such as the gum, lips, oral mucosa and tongue. The composition of the present invention, in an aspect, allows a sustained release of SDF to the tooth's surface during the time period for administration. As such, a sustained release formulation or a release of a relative constant amount of SDF is provided.

Examples of carriers that can be used include copal, denture adhesive, epoxy, silicone, latex, glass ionomer, composite resin, natural adhesives, synthetic adhesives, drying adhesives, emulsion adhesives (e.g., polyvinyl acetate) or any combination thereof, or variants thereof. The composition of the present invention can include carriers that are natural adhesives. Natural adhesives include adhesives made from inorganic mineral sources or biological sources such as vegetable matter, starch (e.g., dextrin), natural resins or from animals e.g. casein or animal glue, or bioadhesive mixtures such as gelatin and starch. Synthetic adhesives can include adhesives such as elastomers, thermoplastics, emulsion or thermosetting adhesives based on polyvinyl acetate, epoxy, polyurethane, cyanoacrylate polymers or any combination thereof. Drying adhesives can include adhesives made from a mixture of ingredients, such as polymers dissolved in a solvent. Emulsion adhesives can consist of dispersions based on polyvinyl acetate

These carriers can be present in an amount that is about 1/2 of what is used in the current water base, or in amounts between about 6% and about 18% by weight. The composition can further include a compound to reduce staining. Such stain reduction compounds include potassium iodide to generate silver Iodide, and/or hexafluorosilicate.

In an embodiment, the SDF composition of the present invention includes e.g., about 5-20% percent by weight of SDF; up to about 10 percent by weight of glycerin; up to about 5 percent by weight of fumed silica; and an amount of varnish or adhesive additives. Examples of additives are flavorants, rosins, gums and alcohols. The composition of the present invention can also contain water.

Preferred rosins are those generally accepted for dental use, and include for example, vinsol rosin or wood rosin, both available from Hercules. The use of rosin is optional, and the amount of such rosin will vary depending upon the desired characteristics of the end product and upon the physical properties of the rosin itself.

Similarly, preferred gums include those generally accepted for dental use, and include for example, Portuguese gum available from Calo, Arentinian gum available from AKZO, and other gums such as Brazilian gum and the like. In a preferred embodiment of the invention, from about 50 to about 75 percent by weight (% W/W) is employed.

Additionally, alcohols for use in the composition of the present invention include those generally accepted for dental use, and include for example, ethanol. From about 20 to 35 percent by weight of ethanol can be used.

Compositions used include gels which preferably display a viscosity of 5 to 20,000 mPas, particularly preferably 100 to 5,000 mPas and quite particularly preferably 1,000 to 5,000 mPas. Viscosities can be measured using methods known in the art. In an instance, viscosities can be measured using a rotational viscometer (measured at 23° C., shearing rate 1 00S-1, conical plate measurement system, cone diameter 20 mm, aperture angle 2⁵, gap 70 I-Im).

The carrier of the oral composition can comprise a combination of a silicone resin and a silicone adhesive, which thus blends a strong and rigid film-forming resin with a soft elastomeric matrix adhesive. In preferred embodiments, the combination of the strong resins and soft adhesives provides an improved flexible film that is able to withstand conditions of the oral environment (e.g., temperature, moisture, pH), as well as physical agitation for an extended period of time. In various embodiments, the combination or ratios of the relative amounts of silicone resin and silicone adhesive can be adjusted to provide one or more of the following: a desired physical and chemical stability of the composition; a desired level of mucoadhesiveness related to the tackiness of the composition; a desired level of flexibility of the composition; a desired level of hardness of the composition; and control over conditions inducing disintegration of the compositions as applied to a tooth surface which further relates to a dissolution rate. For example, to achieve a desired softer consistency, an decreased level of silicone resin as compared to the adhesive will decrease the hardness and rigidity of the composition and allow for a sustained release of SDF. An increase in the level of silicone adhesive as compared to the resin will increase the tackiness level of the composition.

In another embodiment, the carrier of the oral composition can consist of a silica composition that comprises: providing an aqueous reaction medium containing an alkali metal silicate; heating the aqueous reaction medium to a temperature of from about 45° C. to about 65° C.; adding to said aqueous reaction medium an acidulating agent to form a slurry having a pH between about 5.0 to about 5.5; collecting and washing the reaction product which comprises a silica product; drying the silica product; and, optionally, milling said dried silica product.

In an embodiment, the viscosity can be set by adding the varnish former. Preferred varnish formers are cellulose-based, in particular alkyl cellulose-based, varnish formers.

In an embodiment, the carrier can be a gelling agent selected from the group consisting of hydroxyethyl cellulose, hydroxymethyl cellulose and mixtures thereof; 1.4-2.5% of colloidally dispersed silica; 5-20% of glycerol; 0.1-1% of a condensation product of ethylene oxide and polyoxypropylene glycol having a molecular weight of about 8700 and consisting of 20% weight hydrophobic polyoxypropylene glycol moiety of about 1500-1800 molecular weight and about 80% weight polyoxyethylene moiety.

A water-insoluble alkyl cellulose can be used, e.g., alkyl cellulose with a solubility of at most 0.1 g alkyl cellulose per 100 g water at room temperature. In particular, ethyl cellulose can be used.

The required quantity of varnish former depends on the type of varnish former and is preferably such that the varnish has a viscosity within the ranges given above. In the case of the alkyl cellulose preferably used as varnish former, the quantity depends on among other things its chain length and molecular weight and thus on the viscosity formed in the solution. Alkyl cellulose is preferably used in a quantity of 2 to 30 wt.-%, particularly preferably 5 to 20 wt.-%, quite particularly preferably 6 to 12 wt.-%. A person skilled in the art can determine the quantities of other varnish formers required to set the viscosity of the SDF varnish composition.

The composition of the present invention further includes a toothpaste composition. The toothpaste composition includes a SDF composition between about 0.1% and about 10% by weight (e.g., about 0.1% to about 2% by weight), and a carrier that is suitable for toothpaste. For example a toothpaste composition can include, but is not limited to, sorbital, mannitol, and/or xylitol (about 30% and 50% by weight), abrasives (about 15% to about 40% by weight), detergent (about 5% and 15% by weight). In lesser amounts, color, flavoring, glycerine, saccharin, and water are also included.

The composition of the present invention further includes a mouthwash composition. The mouthwash composition includes e.g., a SDF composition about 0.1% and about 10%, and a carrier that is suitable for mouthwash. A mouthwash composition includes e.g., ethanol (about 10% and about 20% by weight), propylene glycol (about 5% and about 15% by weight), glycerol (about 5% and about 20% by weight). In lessor amounts, flavoring, coloring detergents can be present. A buffer and water can also be included in appropriate amounts.

The method of the present invention includes applying the composition to the tooth surface. The method includes subjecting the tooth surface to the SDF composition, as described herein. The SDF composition can be adhered to the tooth surface using an applicator. The applicator can be a tube applicator (e.g., such as in toothpaste or gel), tray, cotton tip, brush, and the like. The tooth surface can be prepared prior to applying the SDF composition. The tooth surface can be cleaned, washed and/or dried to increase adherence of the SDF composition. The SDF composition is applied to all teeth, in the case of preventing or reducing the occurrence of caries. In another embodiment, or in addition, the SDF composition can also be applied to teeth in the beginning stages of caries development as a treatment.

The SDF composition in a gel carrier is applied for a period of between 1 and 10 minutes, and preferable between 2 and 5 minutes. The SDF composition provides a concentration of SDF to the tooth surface in a sustained release fashion, or such that SDF is delivered at a concentration that does not vary by more than about 20% (e.g., not more than between about 1% and 20% variation of SDF administration).

Additionally, the SDF gel composition can be applied to adhesive strips, as in bleaching strips, and can be applied by the patient periodically for 1-10 minutes at a time.

In carrying out the methods of the present invention, the SDF composition can be applied to an individual as a one time treatment, or periodically. In the embodiment in which the SDF composition is administered periodically, administration can occur e.g., daily, weekly, monthly, every 6 months, yearly, or by-yearly. In an instance, as described in the Exemplification, the SDF composition of the present invention was applied only once and significant reduction or arrest in caries occurred, even after 6 months.

The SDF composition in a varnish formation can be applied to the tooth surface, and the composition is cured or is allowed to dry. Once cured, SDF is released to the tooth to treat or reduce caries. The varnish composition can be applied periodically as well. The varnish composition can be applied by a dentist and cured by a curing light, as known in the art. Other varnish composition can be alcohol based and allowed to dry on the tooth surface. In this embodiment, the composition can dry or be cured to remain on one or more teeth for a longer period of time (e.g., about 5 minutes to about 5 years). In this case, the composition of the present invention provides a sustained release over a longer period of time to prevent the development of caries, or to treat carious lesions. The varnish composition can be applied every about 6 months, once 1 year, every 6 months, every 2 years, 3 years, 4 year, or 5 years.

Based on SDF's mechanism of action, bacterial genes can be targeted. As silver kills bacteria and fluoride hardens teeth, then genetic targets can be used to better target bacterial killing. It is believed that the silver and fluoride combine or precipitate to provide longer anti-bacterial and tooth hardening protection.

### Exemplification

6-month Comparison of Silver-diamine-fluoride (SDF) and Fluoride Varnish:

The study included 227 school aged children to compare the effectiveness of a SDF composition and fluoride varnish in the prevention and treatment of caries.

A 12% water-based SDF composition was Fluroplat obtained from Biodinaminca Quimica e Farmaceeutica Ltda, Brazil, Reg # 10298550010. A fluoride composition was used in the study. The fluoride composition comprises silver-diamine fluoride and water.

A ½ of a drop of the 12% by weight SDF composition was applied to the tooth surface of 120 children in the study, while fluoride varnish was applied to 107 children of the study. The compositions were applied once at the time of the initial exam using a tiny brush to any decayed area for 3 min, then the child rinsed.

After 6 months, the data are as follows:

### Baseline Data

TABLE 1
Number of children treated by type of treatment

# Schoolchildren

| Treatment Type | Frequency | Percent |
|---|---|---|
| SDF | 120 | 52.9 |
| Varnish | 107 | 47.1 |
| Total | 227 | 100 |

### 6 Month Data:

TABLE 2
Number of children after 6 months

| Treatment Type | # of School Children | Loss |
|---|---|---|
| SDF | 119 | 1 |
| Varnish | 103 | 4 |
| Total | 222 | 5 |

TABLE 3
SDF treatment

# of School
Children

| Category | Frequency | Percent |
|---|---|---|
| Active Caries | 20 | 16.8 |
| Arrested Caries | 27 | 22.7 |
| Active and Arrested | 69 | 58.0 |
| Caries in same child | | |
| Dental Fillings | 3 | 2.5 |
| Total | 119 | 100.0 |

TABLE 4
Varnish treatment (after 6 months).

# of School
Children

| Category | Frequency | Percent |
|---|---|---|
| Active Caries | 100 | 97.1 |
| Arrested Caries | 0 | 0 |
| Active and Arrested | 0 | 0 |
| Caries in same child | | |
| Dental Fillings | 3 | 2.9 |
| Total | 103 | 100 |

TABLE 5
Calculations

| | | SDF Number Needed To |
|---|---|---|
| | SDF Prevented Fraction | Treat |
| Active Caries | 82.7 | 1.2 |
| Arrested Carries | NA | NA |
| Active & Arrested | NA | NA |

Prevented fraction = [(Varnish % − SDF %)/Varnish %]
Number Needed to Treat = [1/(Varnish % − SDF %)]

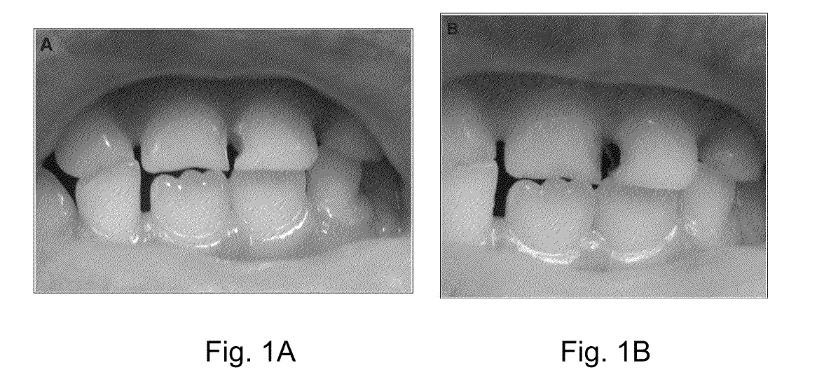

FIG. 1 show photographs of a 5 year older girl treated in the study. FIG. 1A is a clinical photograph prior to SDF treatment and FIG. 1B is a photograph after treatment. The photographs show that only the caries lesion is stained and not the tooth.

This 6-month data indicates that SDF substantially reduces active decay and substantially increases arrested decay, when compared to fluoride varnish. Specifically, these data show active caries in SDF treated patients occurred in only 16.8% after 6 months, as compared to 97.1% in fluoride varnish treated patients, a reduction of by about 80% in active caries. By comparison, the percentage of arrested caries in SDF treated patients was 27%, whereas in fluoride varnish none of the patients had arrested caries. SDF treatment clearly is more effective that the traditional fluoride varnish treatment.

The relevant teachings of all the references, patents and/or patent applications cited herein are incorporated herein by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for arresting caries or reducing the number of active caries lesions in an individual, the method comprises:
a. applying a composition to the tooth surface of the individual for a period of between about 1 and 5 minutes, wherein the composition comprises silver diamine fluoride (SDF) in an amount of between 1% and 40% by weight, and a carrier that provides a sustained release of SDF and adheres to the tooth surface;
b. rinsing said composition from the tooth surface;
wherein caries is arrested or the number of active caries lesions are reduced, as compared to an individual whose tooth surface is not subjected to the SDF composition.

2. The method of claim 1, wherein at least between about 5% and about 30% of caries is arrested.

3. The method of claim 2, wherein at least between about 10% and about 25% of caries is arrested.

4. The method of claim 1, wherein the number of active caries lesions are reduced in a range between about 5% and about 30%.

5. The method of claim 4, wherein the number of active caries lesions are reduced in a range between about 10% and about 25%.

6. The method of claim 1, wherein caries is arrested and active caries lesions are reduced.

7. The method of claim 6, wherein caries is arrested and active caries lesions are reduced at least by a range between about 30% and about 90%.

8. The method of claim 7, wherein caries is arrested and active caries lesions are reduced at least by a range between about 50% and about 70%.

9. The method of claim 1 wherein the carrier comprises natural adhesive, a synthetic adhesive, a drying adhesive, an emulsion adhesive, silicone, copal, glass ionomer, cyanoacrylate, composite resin, latex, epoxy, silicone, water based polyurethane, denture adhesives, or variants thereof or any combination thereof.

10. A method for treating or reducing caries in one or more teeth of an individual, wherein the tooth has a surface, wherein the method comprises:
a. applying a composition to the tooth surface of the individual for a period of between about 1 and 5 minutes, wherein the composition comprises SDF in an amount of between 1% and 40% by weight, and a carrier that provides a sustained release of SDF and adheres to the tooth surface;
b. rinsing said composition from the tooth surface;
wherein caries in one or more teeth of the individual is reduced, as compared to an individual whose tooth surface is not subjected to the SDF composition.

11. The method of claim 10, wherein the SDF composition adheres to the tooth surface and not to the oral mucosa.

12. The method of claim 11, wherein the individual has indications for developing dental caries.

13. The method of claim 12, wherein the individual has braces, has x-ray results showing a decrease in enamel density, or is under the age of 18 years.

14. A method for arresting caries or reducing the number of active caries lesions in an individual, the method comprises:
a. applying a composition to the tooth surface of the individual, wherein the composition comprises silver diamine fluoride (SDF) in an amount of between 1% and 40% by weight, and a varnish that provides a sustained release of SDF and adheres to the tooth surface;
b. curing said composition;
wherein caries is arrested or the number of active caries lesions are reduced, as compared to an individual whose tooth surface is not subjected to the SDF composition.

15. The method of claim 1 wherein the carrier comprises natural adhesive, a synthetic adhesive, a drying adhesive, an emulsion adhesive, silicone, copal, glass ionomer, cyanoacrylate, composite resin, latex, epoxy, silicone, water based polyurethane, denture adhesives, or variants thereof or any combination thereof.

16. A method for treating caries or carious lesions in an individual, the method comprises:
a. applying a composition to the tooth surface of the individual, wherein the composition comprises silver diamine fluoride (SDF) in an amount of between 1% and 40% by weight, and a varnish that provides a sustained release of SDF and adheres to the tooth surface;
b. curing said composition;
wherein caries is treated, as compared to an individual whose tooth surface is not subjected to the SDF composition.

17. A composition that comprises:
a. SDF in an amount between about between about 1% and about 40% by weight; and
b. a carrier that allows for sustained release of SDF.

18. The composition of claim 16, wherein the composition provides a sustained release of SDF and adheres to the tooth surface.

19. The composition of claim 16, wherein the carrier comprises natural adhesive, a synthetic adhesive, a drying adhesive, an emulsion adhesive, silicone, copal, glass ionomer, cyanoacrylate, composite resin, latex, epoxy, silicone, water based polyurethane, denture adhesives, or variants thereof or any combination thereof.

20. The composition of claim 18, wherein the composition comprises SDF in an amount between about 5% and about 20% by weight.

21. A toothpaste or mouthwash composition that comprises:
a. SDF in an amount between about between about 0.1% and about 10% by weight; and
b. a carrier.
